(19) United States
(12) Patent Application Publication
(10) Pub. No.: US 2016/0303331 A1
(43) Pub. Date: Oct. 20, 2016

(54) NEEDLE TIP STORAGE AND REMOVAL DEVICE AND METHODS OF MANUFACTURE THEREOF

(71) Applicant: OWEN MUMFORD LIMITED, Oxford Oxfordshire (GB)

(72) Inventors: Timothy EVANS, Oxford Oxfordshire (GB); Alan HAWKINS, Oxford Oxfordshire (GB); Toby COWE, Oxford Oxfordshire (GB)

(21) Appl. No.: 14/783,630

(22) PCT Filed: Apr. 10, 2014
(86) PCT No.: PCT/GB2014/051125

§ 371 Date: Oct. 9, 2015

(30) Foreign Application Priority Data
Apr. 11, 2013 (GB) 1306601.4

(51) Int. Cl. A61M 5/32; B65B 5/02; B65B 55/08; A61M 5/00; B65B 7/28

(52) U.S. Cl. CPC A61M 5/3205; A61M 5/002; B65B 7/28; B65B 55/08; B65B 5/02; A61M 2207/00

(57) ABSTRACT

A needle tip storage and removal device for use with a needle tip includes a needle hub and a needle extending axially therefrom, which includes a storage compartment formed in a given forming step for containing a needle tip in a sterile environment and for being sealed by an openable seal element, and a needle removal housing for receiving a used needle tip, the housing having been formed in a separate forming step, and being connected to or around the storage compartment.

This invention relates to needle tip storage and removal devices and to methods of manufacture thereof. Many drugs that need to be injected regularly are delivered by a pen injector or the like (such as our Autopen® injector) which has a removable and disposable needle tip (or “pen tip”) which is screwed and unscrewed or otherwise attached and detached by rotary action to the front end of the pen injector to provide a fresh needle for each injection. An example of a typical needle tip is our UniFine® needle tip. This comprises an internally threaded cylindrical hub typically of a plastics material and having a fluted or splined outer cylindrical surface. A needle projects axially forwardly of the hub.

It is known to supply such needle tips in sterile storage containers sealed by means of a foil seal or the like. The storage container has internal splines which engage the external splines on the outer cylindrical surface of the needle tip. The storage container can therefore be used as a spanner or wrench to screw the needle tip into engagement with a threaded portion on the pen injector where this is provided and which can be slid on or off the needle tip. The storage container may provide a similar function in other types of rotary connection. It is preferred that the user, having applied a needle tip to a pen injector and withdrawn the needle tip container to expose the needle tip, should keep the container safe so that it can be used to re-cover the needle. This is so that the container can be used again as a spanner or wrench to unscrew it from the pen injector and then to allow safe disposal of the used needle tip inside the storage container, and also so that the needle is safely covered as the needle tip is being unscrewed.

However, this practice is not always followed with the result that the user may try and remove an unshrouded, used needle tip by hand, with the attendant risk of needle stick injury.

GB2437923 is designed to address this problem and provides a needle tip storage and removal device having opposed, oppositely directed storage and removal compartments, intended to be moulded in one piece in an injection moulding process.

We have found however, this arrangement may be further improved. In particular, in a typical manufacturing facility for such devices, the facility will often be required to switch between insertion, sealing and sterilising a needle tip in a conventional, single container, as well as to be modified to produce runs of needle tips each sealed and sterilised in a storage compartment of a double container of the types shown in GB2437923. This however, requires considerable rejigging of the machinery and can result in a day or so of down time, with the consequent loss in production. In addition, because such devices are normally sterilised by exposure to gamma radiation, and the extent and/or duration of the exposure required is dependant on the mass and volume of material in the device, some of the dosage of radiation is ‘wasted’ on the integrally formed removal compartment, which does not actually need to be sterilised. Also, because the removal tool needs to be designed to have robust puncture resistance, the integrally formed storage and removal compartments are typically made of a material which is puncture resistant. The puncture resistance material tends to be thicker and so increases the amount of material used which is undesirable for a disposable item.

Accordingly, we have designed a needle tip storage and removal device which allows the materials for the storage compartment to be optimised having regard to the requirements of that compartment, that is (maintaining sterility and ease of sterilisation by radiation and the materials of the removal tool to be optimised for its purpose, typically good puncture resistance and good resilience, economy of production and disposability.

In one aspect this invention provides a needle tip storage and removal device for use with a needle tip comprising a needle hub and a needle extending axially therefrom, which includes:

a storage compartment formed in a given forming step for containing a needle tip in a sterile environment and for being sealed by an openable seal element, and

a needle removal housing for receiving a used needle tip, said housing having been formed in a separate forming step, and being connected to or around said storage compartment.

Preferably the hub of said needle tip is generally cylindrical, and said needle tip and said storage compartment are configured to allow said needle tip to be slid into and out of non-rotatable engagement with said removal housing.

Preferably said removal housing is adapted to receive a used needle tip in non-rotatable engagement, when inserted therein.

Preferably said removal housing is configured to receive a needle tip inserted in a direction transverse to said axis.

Preferably said removal housing is adapted to receive a used needle tip when inserted in a direction generally parallel or coincident with said axis.

Preferably said removal housing includes a snap fit engagement arrangement to prevent or resist removal of a used needle tip once inserted into said removal housing.

Preferably said removal housing shrouds the needle of a used needle tip when inserted into said housing, and advantageously said housing fully encloses the needle of said used needle tip.

Preferably the storage container has an outer shape including a larger cylindrical portion and a smaller cylindrical portion interconnected by a bridging portion.

Preferably said removal housing includes at least one ring-shaped portion adapted to surround and secure a portion of the storage container.

Preferably said removal housing includes a spaced ring-shaped portion adapted to surround and secure a further respective spaced portion of said storage compartment.

Preferably each said ring-shaped portion is secured to an adjacent portion of said storage compartment by one or more of: heat welding, ultrasonic welding, adhesive bonding, snap action and mechanical interlock.

Preferably said removal housing includes at least one clip portion adapted to clip around a portion of said storage compartment to secure said removal housing to said storage compartment.

Preferably said removal housing includes two spaced clip portions adapted to clip around respective spaced portions of said storage compartment, to secure said removal housing to said storage compartment.

Preferably said removal housing comprises a larger cylindrical portion merging with a frusto-conical portion which merges with a smaller cylindrical portion.

Preferably said removal housing includes a recess for receiving and locating opposed longitudinal end portions of said storage compartment.

Preferably said removal housing is formed by overmoulding a material over at least a portion of said storage compartment, thereby to provide a removal housing connected around said storage compartment.

Preferably said storage compartment and said removal housing each define respective holding portions for the needle tip such that a needle tip held in the storage compartment is held with its needle extending generally parallel to and facing in the opposite direction to a needle tip held in the removal housing.

In another aspect the invention provides a needle tip storage and removal device for use with a needle tip comprising a cylindrical hub and a needle extending axially therefrom, which comprises a storage compartment formed in a given forming process for containing a needle tip in a sterile environment and for being sealed by an openable seal element, a separately formed outer housing having a recess adapted to receive said storage compartment, and a closure element configured to be moveable between a retracted position in which access to the interior of said storage compartment is allowed, and a closed position in which such access is prevented or inhibited.

Preferably said storage compartment defines an interior recess into which a needle tip may be inserted for non-rotatable engagement, and said outer housing defines a stepped recess having a larger diameter cylindrical portion for receiving said storage compartment and, beyond said larger diameter cylindrical portion, a smaller diameter cylindrical portion into which a needle tip may be inserted for non-rotatable engagement if the larger diameter cylindrical recess is not occupied by a storage compartment.

Preferably said storage compartment includes a removable seal element, and said outer housing and/or said closure element are configured so that the said seal element, if present on said storage compartment, blocks movement of said closure element to its closed position, but when said seal element is absent said closure element may be moved to its closed position.

In another aspect the invention provides a method of producing a needle tip storage and removal device which comprises:

forming a storage compartment in a process step;

forming a removal tool in a separate process step, and

assembling together the storage compartment and the removal tool.

Preferably said storage compartment and said removal tool are formed of different materials.

The method may further include the step of inserting a needle tip into said storage compartment and applying a closure element to said compartment hermetically to seal the needle tip in said storage compartment.

The method may further include the step of sterilising the assembly of the needle tip and the storage compartment.

Preferably said step of sterilisation includes irradiation.

Preferably said removal tool comprises a recess for receiving said needle tip.

Preferably said removal tool is joined to said storage compartment by at least one of the following methods:
ultrasonic welding
mechanical interlock
heat welding, and
adhesion.

Preferably said storage compartment and said removal tool are joined together by forming the removal tool around said storage compartment.

Preferably said removal tool is formed by overmoulding said storage compartment.

Preferably the storage compartment and the removal tool are joined together by forming a removal tool having a recess for receiving the storage compartment and thereafter inserting said storage compartment into said recess.

Whilst the invention has been described above, it extends to any inventive combination or sub-combination of the features set out above, and in the following description, claims or appended drawings.

The invention may be performed in various ways and, by way of example only, various embodiments thereof will now be described in detail, reference being made to the accompanying drawings in which:

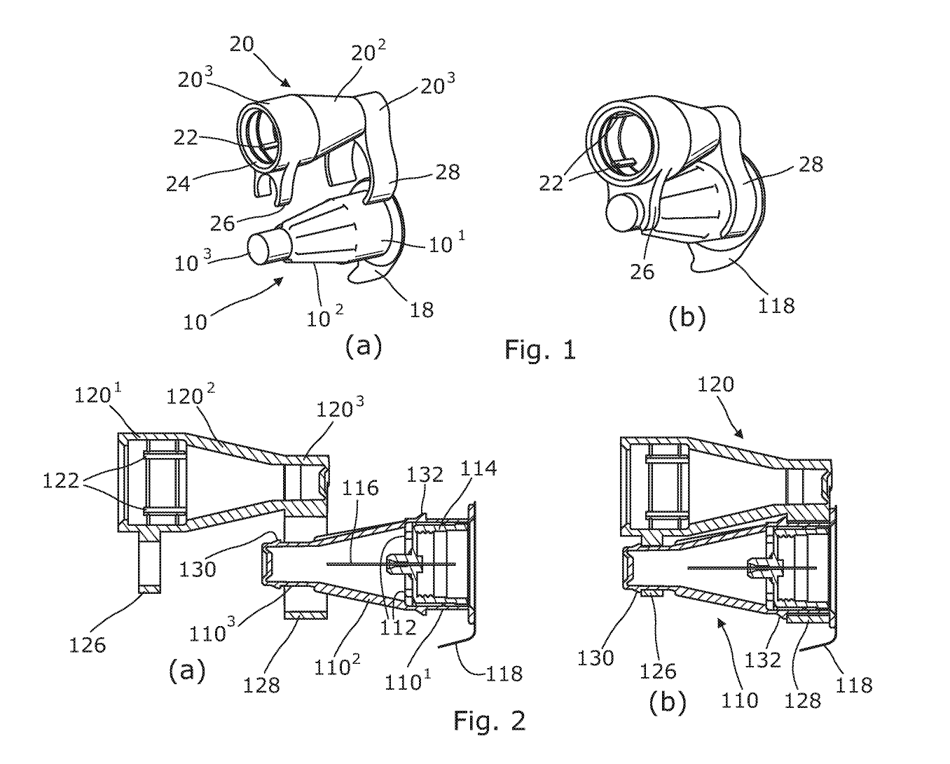

FIGS. 1(a) and (b) are perspective views of a needle tip storage and removal device, with the main components before and after assembly respectively;

FIGS. 2(a) and (b) are section views of a second embodiment of needle tip storage and removal device before and after assembly respectively;

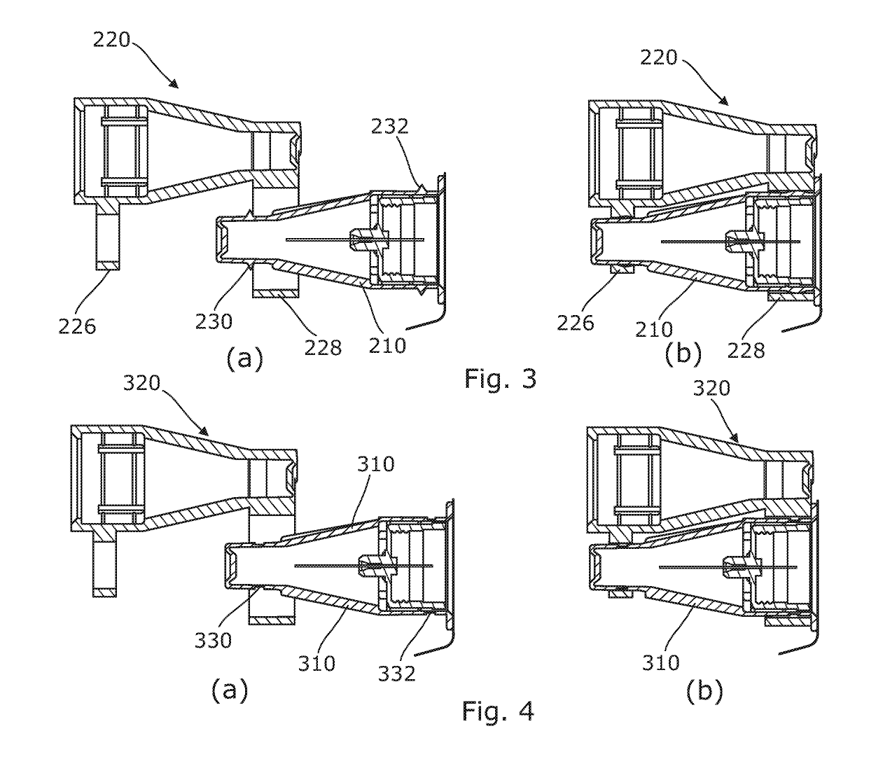

FIGS. 3(a) and (b) are section views of a third embodiment of needle tip storage and removal device before and after assembly respectively;

FIGS. 4(a) and (b) are section views of a fourth embodiment of needle tip storage and removal device before and assembly respectively;

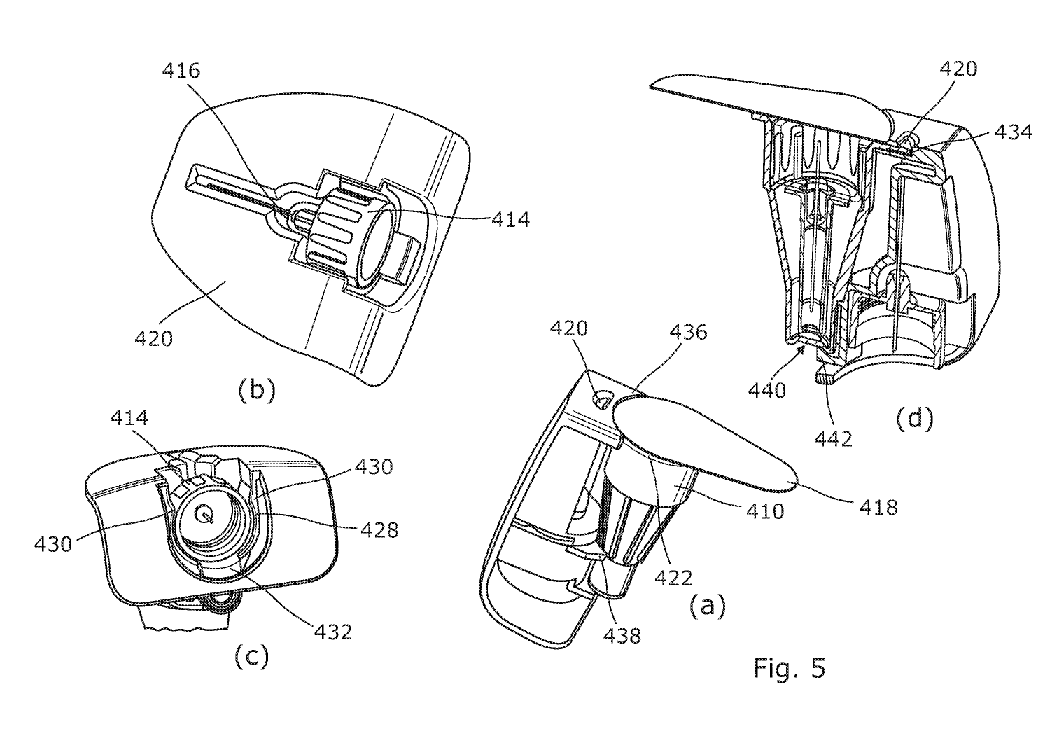

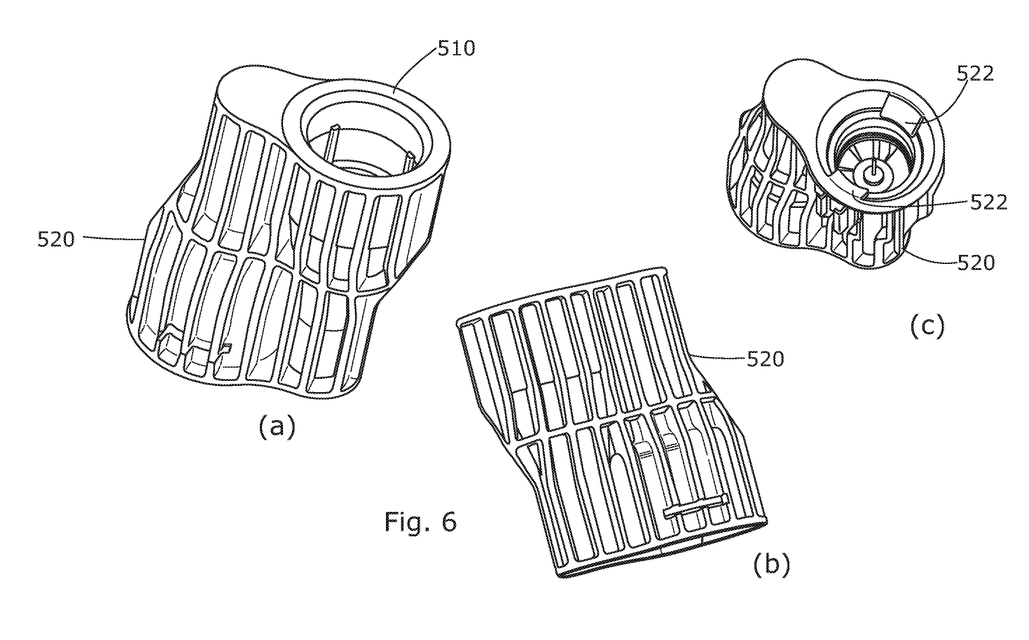

FIGS. 5(a) to (d) are views of a fifth embodiment of needle tip storage and removal device;

FIGS. 6(a) to (c) are views of a sixth embodiment of needle tip storage and removal device;

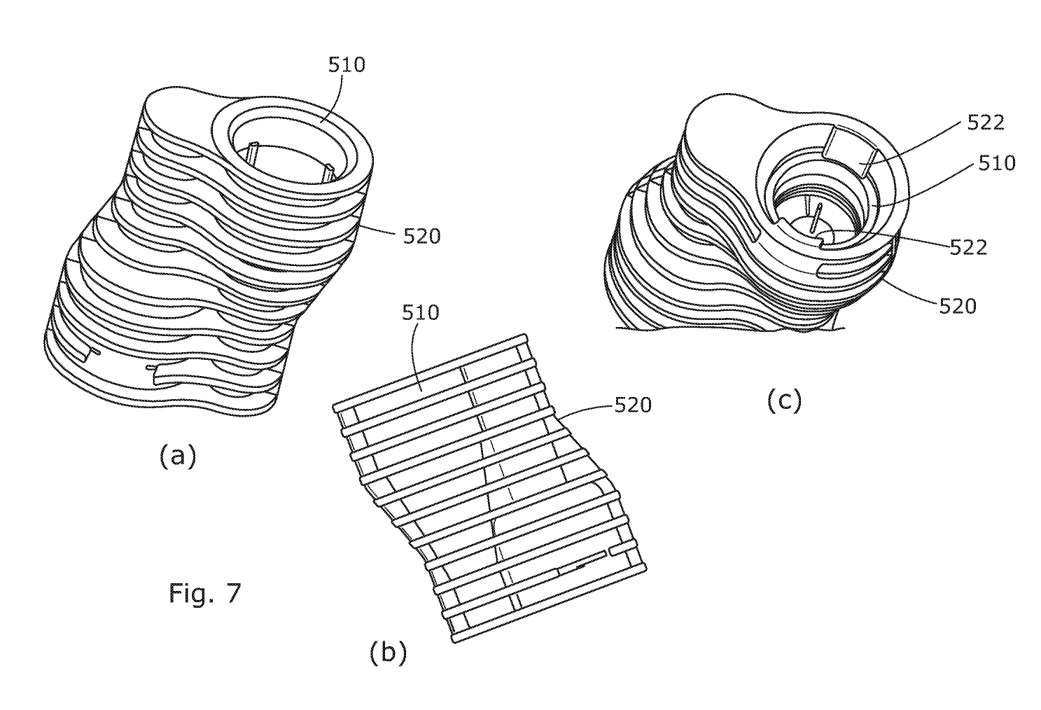

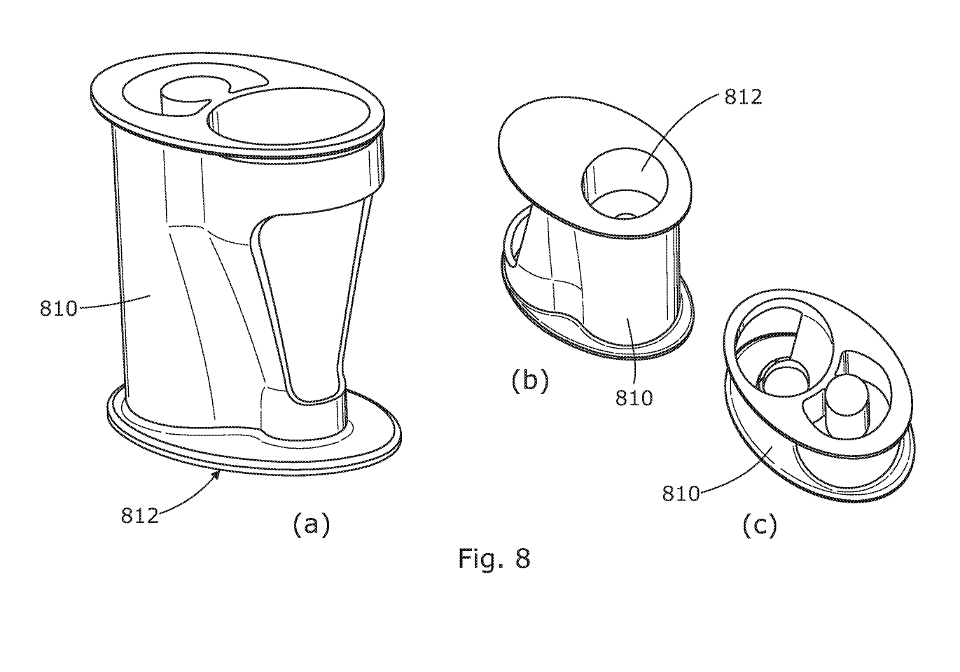

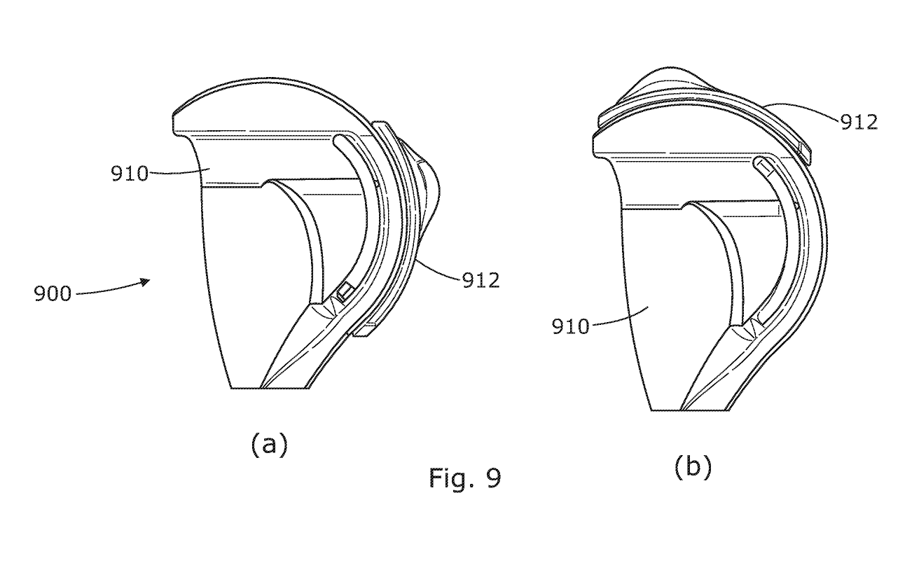

FIGS. 7(a) to (c) are views of a seventh embodiment of needle tip storage and removal device;

FIGS. 8(a) to (c) are views of an eighth embodiment of needle tip storage and removal device, prior to insertion of the needle tip storage container;

FIGS. 9(a) and (b) are views of a ninth embodiment of needle tip storage and removal device in the as supplied and locked conditions respectively;

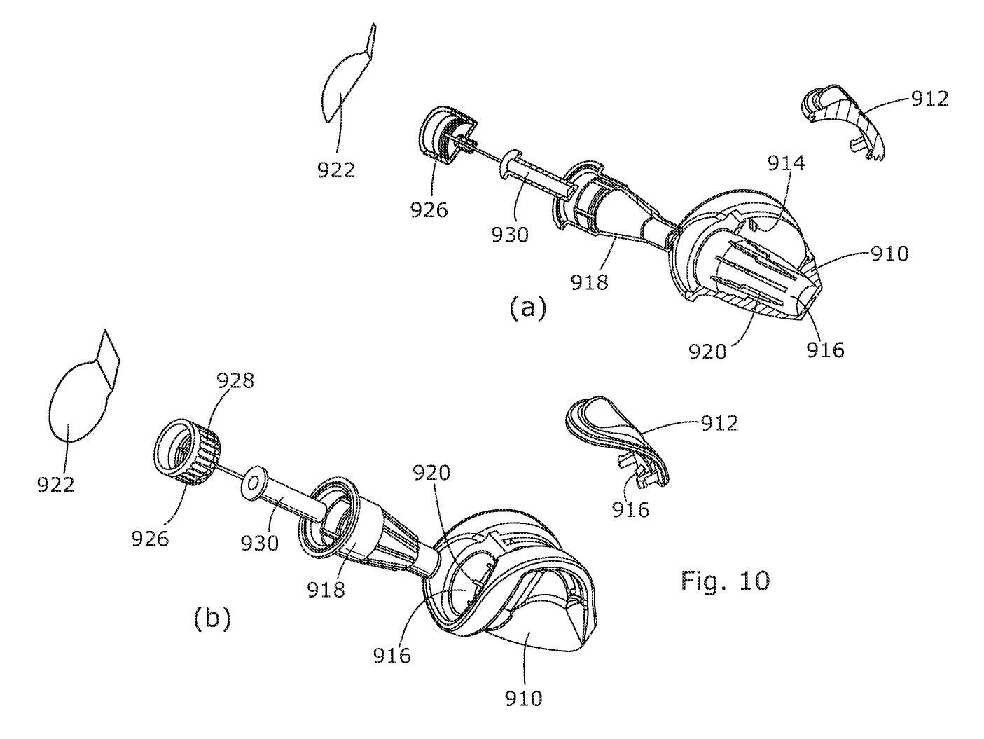

FIGS. 10(a) and (b) are exploded section and perspective views respectively of the ninth embodiment of needle tip storage and removal device, and

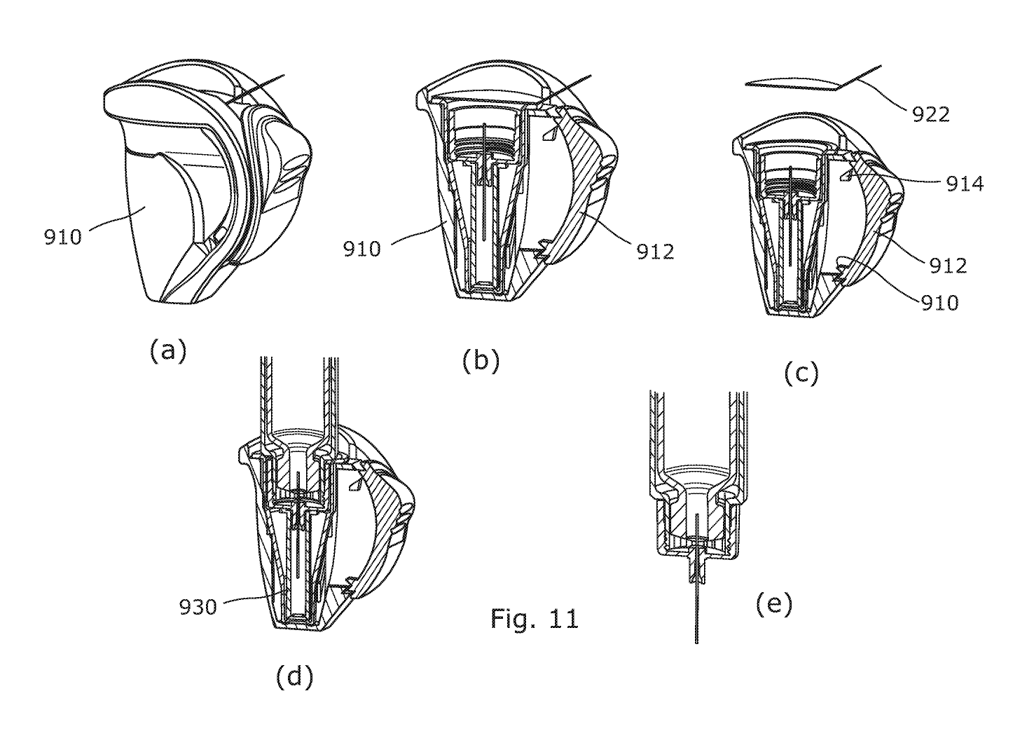

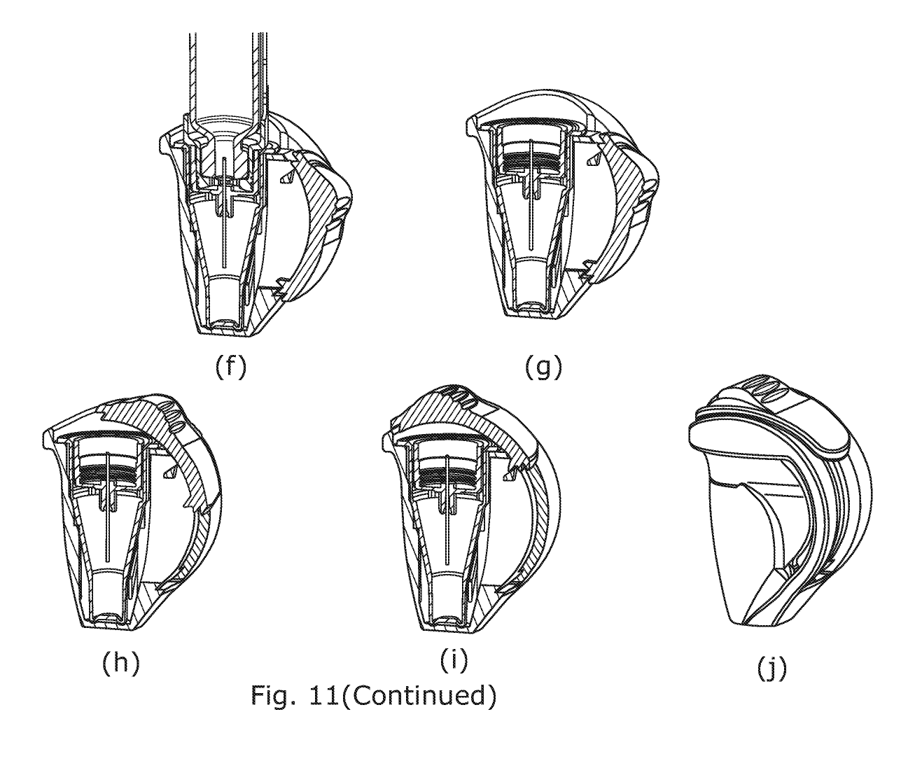

FIGS. 11(a) to (j) are successive views showing use of the ninth embodiment of needle tip storage and removal device.

The various embodiments described herein provide needle tip storage and removal devices made up of a storage container or compartment containing a needle tip and hermetically sealed by a removable foil or the like, the needle tip container then being assembled with a removal housing which is or has been formed in a separate forming step. This means that the needle tip storage and removal device may be made by taking a pre-existing needle tip container sealed with the needle tip inside it, and then assembling it with the removal housing. In this way, the moulding of the storage container, loading of the needle tip and subsequent sealing of the container may be optimised for subsequent sterilisation process by gamma irradiation prior to assembly with the removal housing. The sterile needle tip storage container, with needle tip inside, may then be assembled in a sterile manner with the removal housing.

In the embodiments below, the needle tip storage container is made up of a relatively large diameter portion 10¹, which merges with a frustoconical portion 10² which itself merges with a closed tip portion 10³ of relative small diameter, as seen for example in FIGS. 1(a), 5(a) and 10(b). Internally, the larger cylindrical portion defines an internal cylindrical wall with splines 12 equi-spaced around it, the splines being designed to allow the needle tip hub to be slid into and out of non-rotatable engagement with the needle tip storage container. In this example, the needle tip has a double ended needle (see FIG. 2(a)). The needle tip storage container 10 is sealed by a removable foil 18. In the first embodiment, the needle tip removal housing 20 is formed in a separate process and from, typically, different materials but has a similar overall shape and internal shape as that of the needle tip storage container 10. Thus the needle tip removal housing 20 has a relatively large diameter portion 20¹ that merges with a frustoconical portion 20² which itself merges with a cylindrical tip portion 20³. Internally, the large cylindrical portion 20¹ is provided with splines 22 into which a used needle tip may be slid in non-rotatable engagement. The open end of the larger diameter cylindrical portion 20¹ is provided with an internal snap rib 24 designed to lock a used needle tip in the removal housing 20 when it is fully pushed home.

Projecting transversely from the large cylindrical portion 20¹ and the small cylindrical portion 20³ are respective small and large clip portions 26, 28 respectively defining resilient clip recesses which allow the needle tip storage container and the removal housing to be clipped together as shown in FIG. 1(b). The clipping action may be permanent or reversible.

Referring now to FIG. 2, many features of the second embodiment are similar to those of the first embodiment and will be given similar reference numbers but incremented by 100. In this embodiment, the needle tip removal housing is provided with laterally extending small and large ring portions 126 and 128 respectively. The needle tip storage container is provided with small and large diameter snap rings 130, 132 on the external surface of the small and large diameter cylindrical portions 110³ and 110¹. The needle tip storage container and the needle tip removal housing may be assembled together by disposing them in the position of FIG. 2(a) and then bringing them together so that the ribs 130, 132 squeeze through and snap through the ring shaped portions 126 and 128, as seen in FIG. 2(b).

Referring now to FIG. 3, many features of the third embodiment are similar to those of the second embodiment and will be given similar reference numbers but incremented by 100. In this embodiment, the small and large diameter portions of the needle tip storage container 210 are formed with annular ribs 230 and 232. The needle tip storage container 210 and the needle tip removal housing 220 are positioned as shown in FIG. 3 and then pushed together so that the annular ribs 230 and 232 contact and pass into the inner cylindrical surface of rings 226, 228 on the needle tip removal housing 220. An ultrasonic welding process is then applied to cause the ribs 230 and 232 to fuse or melt with surrounding ring portion 226, 228 permanently to secure the needle tip storage container and the needle tip removal housing together.

Referring now to FIG. 4, many features of the fourth embodiment are similar to those of the third embodiment and will be given similar reference numbers but incremented by 100. In this embodiment, the needle tip storage container 310 is provided with annular recesses 330 and 332 in its small and large diameter portions respectively. The needle tip removal housing 320 is identical to that of the fourth embodiment and the needle tip storage container and the needle tip removal housing are assembled together by applying an adhesive to the grooves 330 and 332 and then assembling the two together as shown in FIG. 4(b).

Referring now to FIG. 5, in this embodiment a needle tip storage container 410 similar to that of the previous embodiment, containing a needle tip of the type described above and sealed by a foil 418 is provided with a tab 420 that extends transversely from a flange 422 provided at the open end of the larger cylindrical portion of the needle tip storage container. The flange 422 provides a planar radial surface to which the foil 418 is heat-sealed. The tab 420 is formed integrally in the same moulding process as the main portion of the needle tip storage container 410.

In this embodiment, the needle tip removal housing is based on our existing UniGuard® device, as described in WO 2005/102424, the contents of which are incorporated herein by reference. The needle tip removal housing 426 has an opening 428 of outline generally matching and adapted to receive the hub 414 and needle 416 of a needle tip when inserted laterally. The opening includes a U-shaped cradle portion 428 which has two abutments 430 past which the needle hub snaps when inserted laterally. Once in its fully home position, the needle hub engages splines (not shown) on the interior of the U-shaped portion 428 to engage it non-rotatably. If required, there may be a two stage engagement action whereby the user initially inserts the needle hub laterally and then pushes it axially (in the direction of the needle) to a fully home position. In this latter position, the needle hub may snap past a tongue 432 to hold it against reverse axial movement. As seen particularly in FIGS. 5(a) and 5(d), the needle removal housing 426 is provided with a recess 434 into which the tab 420 on the flange of the needle tip storage container be snapped irreversibly. Snapping the tab 420 into the recess 434 holds the needle tip storage container closely against the underside of the needle tip removal housing 426, with portions thereof cradled by supports 436, 438 and the smaller diameter end of the needle housing is provided with a dished portion 440 over into the edge of which hooks a protrusion 442 to hold the needle tip storage container and the needle tip removal housing securely together.

Referring now to the embodiments of FIGS. 6 and 7, here a two-shot or overmoulding process is used to initially form a needle tip storage container 510 of similar form to those of the earlier embodiments which can then be fitted with a needle tip sealed and then sterilised. In the second moulding step, using a different plastics material, a needle tip removal housing 520 is moulded which has a main needle tip receiving portion with an integral frame structure embracing and connecting the needle tip storage container. The needle tip removal portion may include flexible tabs 522 to prevent removal of a used needle tip once inserted into the needle tip removal portion. As with the other embodiments, the needle tip removal portion has a recess configured non-rotatably to receive a used needle tip. In FIG. 6, the main frame elements encasing the needle tip storage container run generally longitudinally, whereas in the arrangement of FIG. 7, they run generally circumferentially.

Referring now to FIG. 8, in this embodiment a needle tip removal housing 810 is provided with a needle tip removal portion 812 designed non-rotatably to receive a used needle tip as before. Adjacent to the needle tip removal portion there is a shaped recess 814 designed to receive by a snap fit a needle tip storage container. As previously, therefore, each of the needle tip removal housing and a needle tip storage container may be separately formed and then assembled together to form the final product.

Referring now to the embodiment illustrated in FIGS. 9 to 11, here the needle tip storage and removal device is made up by assembling a needle tip within the needle tip storage container to provide sterile primary packaging as previously, with that assembly then being fitted into a disposal system 900, thereby allowing broader material selection for the disposal system and reducing processing costs. The needle disposal system comprises a housing 910 with a moveable 912 lid that can be moved from an open position, in which access to the needle tip storage container is allowed, and a closed position in which access to the needle storage container is obstructed. The device may use any suitable form of closing movement but, in the embodiment of FIGS. 9 to 11, an arcuate sliding movement is used to slide a cover from the open position shown in FIG. 9(a) to the closed position shown in FIG. 9(b). The cover includes a latching mechanism 914, 916 to latch it in its closed position.

Referring now to FIG. 10, the housing 910 has a recess 916 designed to receive the needle tip storage container 918. The needle tip storage container may be an interference fit within the recess or it may snap into place. As seen more particularly in FIG. 10(a), the recess is provided with stepped internal radial ribs 920 which co-operate with formations 922 on the outside of the needle tip storage container to prevent rotation there between. The latching mechanism for locking the cover 912 in its closed position comprises a tooth 914 on the housing which co-operates with the corresponding flexible tooth on the cover 916. The device of FIG. 10 is designed so that, when a needle storage container 918 is fitted into the recess, the foil 922 on the needle storage container 918 obstructs movement of the cover 912 towards its closed position, so that the cover can only be slid closed once the foil has been removed.

Referring again to FIG. 10(a), the radial ribs 920 are stepped inwardly and designed so that, in the event that the needle tip storage container should somehow become dislodged from the disposal system, as a failsafe arrangement the recess can slideably receive a non-rotatable fashion the needle hub 926, with the inwardly stepped ribs 920-co-operating with the splines 928 on the hub.

Referring to FIG. 11, when the device is as supplied, the cover 912 is its open position and prevented from moving to the closed position by the tab of the foil seal 922. The user prepares the device by removing the foil seal 922 and then inserting a pen and screwing it on to the needle to collect the needle. The user then withdraws the pen from the device and then removes an inner needle shield 930 to expose the needle ready for an injection (FIG. 11(e)). After the injection, the user replaces the needle hub into the needle storage container and unscrews the pen. This can be seen in FIG. 11(f). As an additional feature, should the primary packaging have fallen out, the pen can be inserted deeper into the disposal system to engage the needle hub with the radial splines. Having removed the pen, the cover may be slid around the body to cover the needle with the tooth on the cover snapping past the tooth on the housing to lock it in place. The used needle is then in a safe condition ready for disposal.

1. A needle tip storage and removal device for use with a needle tip comprising a needle hub and a needle extending axially therefrom, which includes:
a storage compartment formed in a given forming step for containing a needle tip in a sterile environment and for being sealed by an openable seal element, and
a needle removal housing for receiving a used needle tip, said housing having been formed in a separate forming step, and being connected to or around said storage compartment.

2. A needle tip storage and removal device according to claim 1, wherein the hub of said needle tip is generally cylindrical, and said needle tip and said storage compartment are configured to allow said needle tip to be slid into and out of non-rotatable engagement with said removal housing.

3. A needle tip storage and removal device according to claim 1, wherein said removal housing is adapted to receive a used needle tip in non-rotatable engagement, when inserted therein.

4. A needle tip storage and removal device according to claim 3, wherein said removal housing is configured to receive a needle tip inserted in a direction transverse to said axis.

5. A needle tip storage and removal device and removal device according to claim 3, wherein said removal housing is adapted to receive a used needle tip when inserted in a direction generally parallel or coincident with said axis.

6. A needle tip storage and removal device according to claim 1, wherein said removal housing includes a snap fit engagement arrangement to prevent or resist removal of a used needle tip once inserted into said removal housing.

7. A needle tip storage and removal device according to claim 1, wherein said removal housing shrouds the needle of a used needle tip when inserted into said housing.

8. A needle tip storage and removal device according to claim 7, wherein said housing fully encloses the needle of said used needle tip.

9. A needle tip storage and removal device according to claim 1, wherein the storage container has an outer shape including a larger cylindrical portion and a smaller cylindrical portion interconnected by a bridging portion.

10. A needle tip storage and removal device according to claim 1, wherein said removal housing includes at least one ring-shaped portion adapted to surround and secure a portion of the storage container.

11. A needle tip storage and removal device according to claim 10, wherein said removal housing includes a spaced ring-shaped portion adapted to surround and secure a further respective spaced portion of said storage compartment.

12. A needle tip storage and removal device according to claim 10, wherein each said ring-shaped portion is secured to an adjacent portion of said storage compartment by one or more of: heat welding, ultrasonic welding, adhesive bonding, snap action and mechanical interlock.

13. A needle tip storage and removal device according to claim 1, wherein said removal housing includes at least one clip portion adapted to clip around a portion of said storage compartment to secure said removal housing to said storage compartment.

14. A needle tip storage and removal device according to claim 13, wherein said removal housing includes two spaced clip portions adapted to clip around respective spaced portions of said storage compartment, to secure said removal housing to said storage compartment.

15. A needle tip storage and removal device according to claim 1, wherein said removal housing comprises a larger cylindrical portion merging with a frusto-conical portion which merges with a smaller cylindrical portion.

16. A needle tip storage and removal device according to claim 1, wherein said removal housing includes a recess for receiving and locating opposed longitudinal end portions of said storage compartment.

17. A needle tip storage and removal device according to claim 1, wherein said removal housing is formed by overmoulding a material over at least a portion of said storage compartment, thereby to provide a removal housing connected around said storage compartment.

18. A needle tip storage and removal device according to claim 1, wherein said storage compartment and said removal housing each define respective holding portions for the needle tip such that a needle tip held in the storage compartment is held with its needle extending generally parallel to and facing in the opposite direction to a needle tip held in the removal housing.

19. A needle tip storage and removal device for use with a needle tip comprising a cylindrical hub and a needle extending axially therefrom, which comprises a storage compartment formed in a given forming process for containing a needle tip in a sterile environment and for being sealed by an openable seal element, a separately formed outer housing having a recess adapted to receive said storage compartment, and a closure element configured to be moveable between a retracted position in which access to the interior of said storage compartment is allowed, and a closed position in which such access is prevented or inhibited.

20. A needle tip storage and removal device according to claim 19, wherein said storage compartment defines an interior recess into which a needle tip may be inserted for non-rotatable engagement, and said outer housing defines a stepped recess having a larger diameter cylindrical portion for receiving said storage compartment and, beyond said larger diameter cylindrical portion, a smaller diameter cylindrical portion into which a needle tip may be inserted for non-rotatable engagement if the larger diameter cylindrical recess is not occupied by a storage compartment.

21. A needle tip storage and removal device according to claim 19, wherein said storage compartment includes a removable seal element, and said outer housing and/or said closure element are configured so that the said seal element, if present on said storage compartment, blocks movement of said closure element to its closed position, but when said seal element is absent said closure element may be moved to its closed position.

22. A method of producing a needle tip storage and removal device which comprises:
forming a storage compartment in a process step;
forming a removal tool in a separate process step, and
assembling together the storage compartment and the removal tool.

23. A method according to claim 22 wherein said storage compartment and said removal tool are formed of different materials.

24. A method according to claim 22, which includes the step of inserting a needle tip into said storage compartment and applying a closure element to said compartment hermetically to seal the needle tip in said storage compartment.

25. A method according to claim 24, which further includes the step of sterilising the assembly of the needle tip and the storage compartment.

26. A method according to claim 25, wherein said step of sterilisation includes irradiation.

27. A method according to claim 22, wherein said removal tool comprises a recess for receiving said needle tip.

28. A method according to claim 22, wherein said removal tool is joined to said storage compartment by at least one of the following methods:
ultrasonic welding
mechanical interlock
heat welding, and
adhesion.

29. A method according to claim 22, wherein said storage compartment and said removal tool are joined together by forming the removal tool around said storage compartment.

30. A method according to claim 29, wherein said removal tool is formed by overmoulding said storage compartment.

31. A method according to claim 22, wherein the storage compartment and the removal tool are joined together by forming a removal tool having a recess for receiving the storage compartment and thereafter inserting said storage compartment into said recess.
